(19) United States
(12) Patent Application Publication
(10) Pub. No.: US 2019/0259152 A1
(43) Pub. Date: Aug. 22, 2019

(54) PORTABLE RADIOGRAPHIC APPARATUS AND RADIOGRAPHIC IMAGE CORRECTION METHOD

(71) Applicant: Konica Minolta, Inc., Tokyo (JP)

(72) Inventor: Masashi KONDO, Tokyo (JP)

(21) Appl. No.: 16/256,531

(22) Filed: Jan. 24, 2019

(30) Foreign Application Priority Data
Feb. 22, 2018 (JP) 2018-029254

(51) Int. Cl. G06T 7/00; A61B 6/00; H04N 5/361

(52) U.S. Cl. CPC G06T 7/0012; A61B 6/548; H04N 5/361; A61B 6/4405; A61B 6/40; A61B 6/5211

(57) ABSTRACT

A portable radiographic apparatus 1 includes a hardware processor that sequentially acquires a plurality of image data by repeating charge accumulation and release by pixels r and image data readout by a readout integrated circuit 16 a plurality of times, builds a temporal change model of a signal value based on a plurality of dark charge image data O acquired before a start of radiographing, calculates a plurality of correction values based on the built temporal change model, and applies the respective corresponding correction values to image data of a plurality of radiographic images acquired during radiographing of a subject to correct the image data of the respective radiographic images.

### BACKGROUND

### Technological Field

The present invention relates to a portable radiographic apparatus and a radiographic image correction method.

### Description of the Related Art

Radiographic apparatuses capable of generating image data of radiographic images generally include a sensor substrate with pixels disposed thereon so as to be two-dimensionally distributed, the pixels being capable of generating charge upon reception of radiation and accumulating the charge, and a readout integrated circuit (ROIC) connected to the sensor substrate, the readout integrated circuit reading out a signal value of each pixel based on an amount of charge released from the pixel.

Also, from among these radiographic apparatuses, there are ones capable of performing moving image radiographing (also referred to as “serial radiographing”) in which accumulation of charges and readout of signal values are repeated a plurality of times in a short period of time, based on a single radiographing start instruction (for example, pressing of an exposure switch).

It is known that in a radiographic apparatus capable of performing moving image radiographing, during moving image radiographing, since a high load is imposed on the readout integrated circuit over a relatively long period of time, the readout integrated circuit generates heat and a temperature of the readout integrated circuit continuously increases. Then, upon an increase in temperature of the readout integrated circuit, an offset component contained in a read-out signal value increases. Hereinafter, temporal change in offset component in a signal value accompanying an increase in temperature of a readout integrated circuit is referred to as “offset drift”. An offset drift is likely to cause an artifact to be generated when a taken moving image is analyzed.

For a countermeasure for such offset drift, for example, a technique that models temporal change of an offset component in a signal value of each pixel during a period from a radiographing start instruction to exposure and corrects a signal value of each pixel based on the model has been proposed (see Japanese Patent No. 5460103).

However, the technique described in Japanese Patent No. 5460103 is intended for stationary radiographic apparatuses. Stationary radiographic apparatuses each include means for stabilizing a temperature of a readout integrated circuit and do not cause an offset drift to be generated so much. Therefore, correction of radiographic images can sufficiently be performed using the conventional correction technique.

On the other hand, portable radiographic apparatuses have large restrictions in size and weight of the apparatuses and thus have difficulty in including means for stabilizing a temperature of a readout integrated circuit. In other words, in portable radiographic apparatuses, an offset drift is pronounced in comparison with stationary radiographic apparatuses. Therefore, with a conventional technique such as that described in Japanese Patent No. 5460103, accuracy of correction is low and it is difficult to correct an offset component to an extent that diagnosis is not affected by the offset component.

### SUMMARY

The present invention has been made in view of the above point and an object of the present invention is to enable correcting with high accuracy an offset component in each of frames forming a moving image even if an offset drift occurs when moving image radiographing is performed in a portable radiographic apparatus that can perform moving image radiographing and can easily be carried around by a user.

To achieve at least one of the abovementioned objects, according to a first aspect of the present invention, a radiographic apparatus reflecting one aspect of the present invention comprises: a radiation detector including a substrate and a plurality of pixels capable of accumulating and releasing an amount of charge, the amount corresponding to an intensity of radiation received, the plurality of pixels being provided so as to be two-dimensionally distributed on a surface of the substrate; a readout integrated circuit that reads out image data based on the amount of charge released from each of the pixels; and a hardware processor that sequentially acquires a plurality of image data by repeating charge accumulation and release by the pixels and image data readout by the readout integrated circuit a plurality of times, builds a temporal change model of a signal value based on image data of a plurality of dark charge images acquired before a start of radiographing, calculates a plurality of correction values based on the built temporal change model, and applies the respective corresponding correction values to image data of a plurality of radiographic images acquired during radiographing of a subject to correct the image data of the respective radiographic images.

According to a second aspect of the present invention, a radiographic image correction method reflecting one aspect of the present invention comprises: sequentially acquiring image data of a plurality of dark charge images by repeating image data generation by a radiographic apparatus capable of generating image data of a radiographic image according to radiation received, a plurality of times before a start of radiographing; building a temporal change model of a signal value based on the acquired image data of the plurality of dark charge images; calculating a plurality of correction values based on the built temporal change model; sequentially acquiring image data of a plurality of radiographic images by repeating image data generation by the radiographic apparatus a plurality of times during radiographing of a subject; and applying the respective corresponding correction values to the acquired image data of the plurality of radiographic images to correct the image data of the respective radiographic images.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a perspective view illustrating an outer appearance of a portable radiographic apparatus according to the present embodiment;

FIG. 2 is a plan view illustrating an example configuration of a sensor substrate;

FIG. 3 is a block diagram illustrating an equivalent circuit of a portable radiographic apparatus;

FIG. 4 is a block diagram illustrating an equivalent circuit for one pixel included in a detector;

FIG. 5 is a timing chart illustrating voltage change in each of a charge reset switch, a pulse signal, a certain scanning line and a next scanning line in image data readout processing;

FIG. 6 is a timing chart illustrating, e.g., timings of application of an on-state voltage to each scanning line where radiographing is performed in a cooperation scheme;

FIG. 7 is a timing chart illustrating, e.g., timings of application of an on-state voltage to each scanning line where radiographing is performed in a non-cooperation scheme;

FIG. 8 is a diagram illustrating an example configuration of a noise detector;

FIG. 9 is a diagram illustrating that a readout circuit with no signal line connected thereto is used as a readout circuit of a noise detector;

FIG. 10 is a diagram illustrating that the noise detector in FIG. 8 is configured by noise detectors 31A to 31C;

FIG. 11 is a diagram illustrating that read-out image data contains noise data and illustrating corrected image data Dc;

FIG. 12 is a diagram illustrating that data detected by a noise detector contains an offset component in the noise detector in addition to noise data;

FIG. 13 is a diagram illustrating another example configuration of a noise detector;

FIG. 14 is a diagram illustrating that in the noise detector in FIG. 13, an offset component in the noise detector is multiplied by 1/W in comparison with the noise detector in FIG. 8;

FIG. 15 is a diagram illustrating that in the noise detector in FIG. 13, an effect of the offset component in the noise detector is reduced in corrected image data;

FIG. 16 is a graph indicating temporal change of an offset component in a noise detector;

FIG. 17 is a diagram illustrating an effect of an offset drift;

FIG. 18 is a diagram signal value illustrating a method for building a temporal change model;

FIG. 19 is a flowchart illustrating a flow of moving image radiographing using the portable radiographic apparatus according to the present embodiment;

FIG. 20A is a graph indicating a relationship between the number of frames taken and signal values of pixels in each frame before and after correction where a conventional image correction method is used; and

FIG. 20B is a graph indicating a relationship between the number of frames taken and signal values of pixels in each frame before and after where an image correction method according to the present embodiment is used.

### DETAILED DESCRIPTION OF EMBODIMENTS

Hereinafter, one or more embodiments of the present invention will be described with reference to the drawings. However, the scope of the invention is not limited to the disclosed embodiments.

### [Radiographic Apparatus]

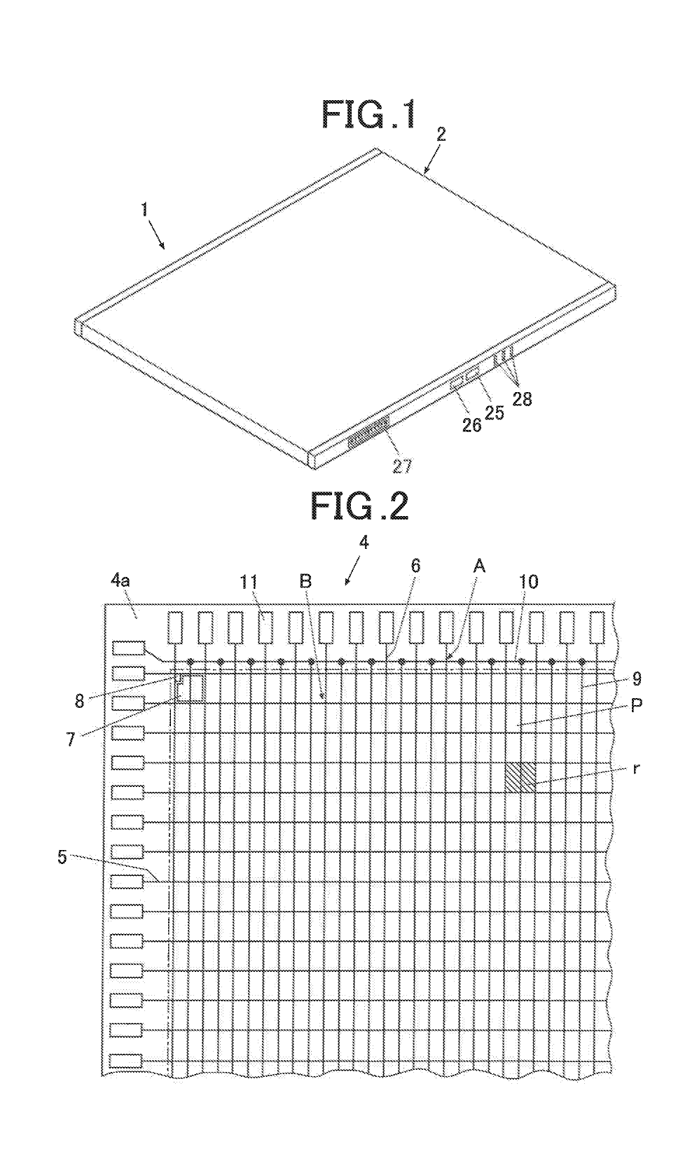

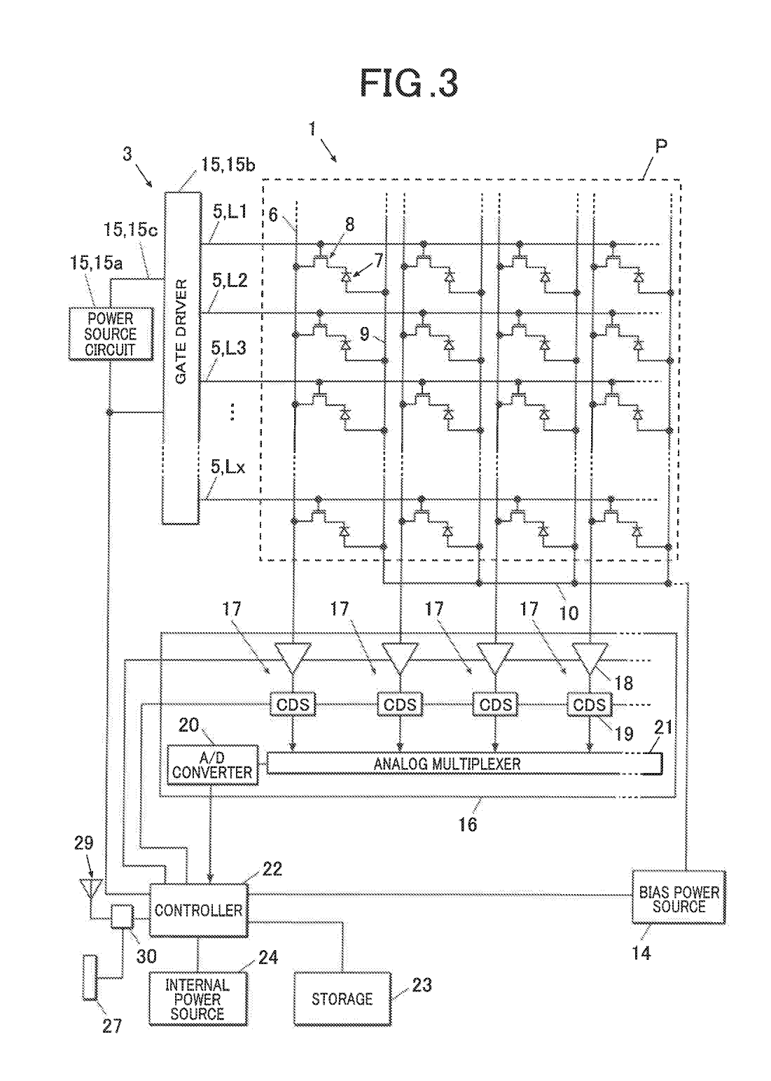

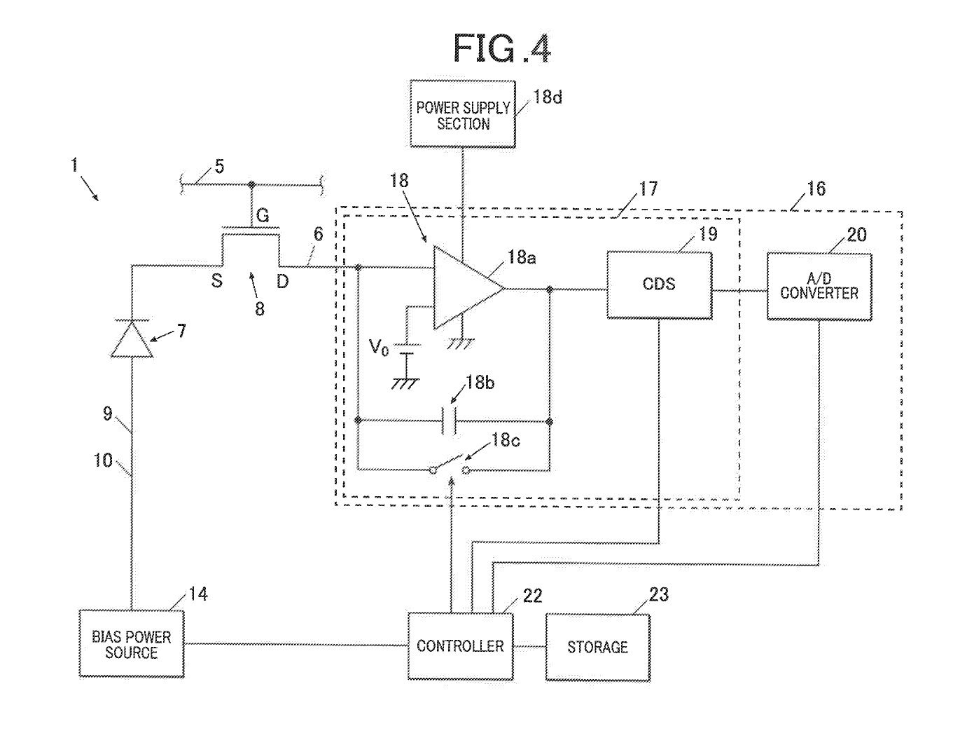

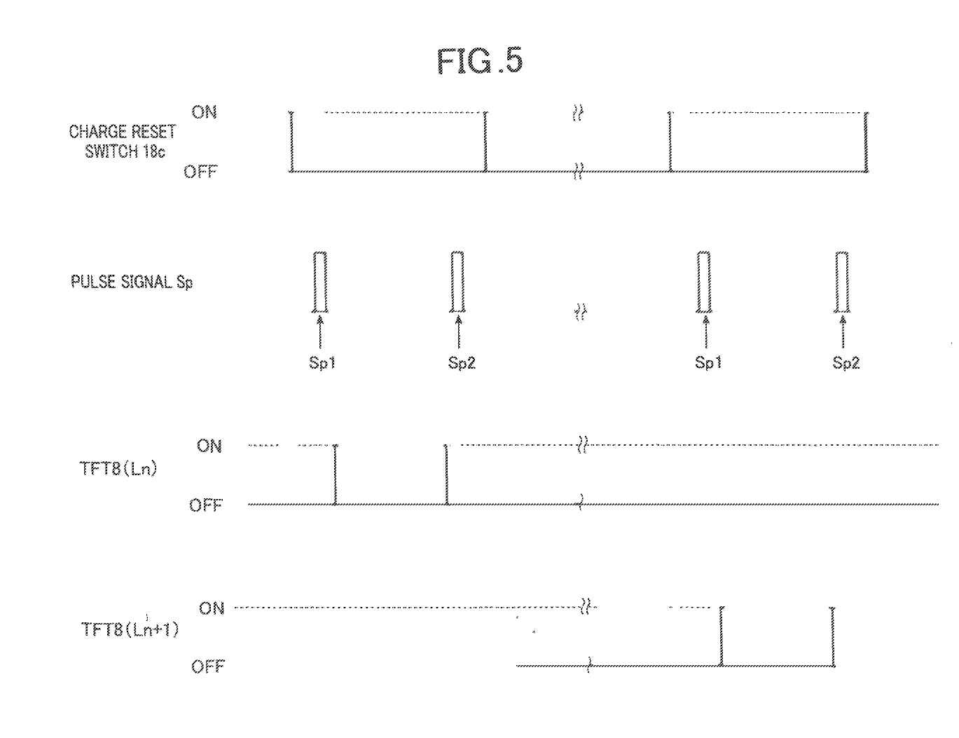

FIG. 1 is a perspective view illustrating an outer appearance of a portable radiographic apparatus according to the present embodiment (hereinafter “radiographic apparatus 1”), and FIG. 2 is a plan view illustrating an example configuration of a sensor substrate 4 incorporated in the portable radiographic apparatus. FIG. 3 is a block diagram illustrating an equivalent circuit of the radiographic apparatus 1, and FIG. 4 is a block diagram illustrating an equivalent circuit for one pixel. FIGS. 5 to 7 are timing charts each illustrating operation of the radiographic apparatus 1.

As illustrated in FIG. 1, the radiographic apparatus 1 according to the present embodiment has a panel-like shape and is of a portable type (also referred to as a “cassette type”) that enables a user to easily carry around.

A power supply switch 25, a selector switch 26, a connector 27, indicators 28, etc., are disposed at a side surface on one side of a casing 2 of the radiographic apparatus 1. An antenna 29 (see FIG. 3) for wireless external communication is provided on a side surface on the opposite side of the casing 2.

Also, in the radiographic apparatus 1, a radiation detector 3 is housed inside the casing 2.

The radiation detector 3 includes, e.g., a sensor substrate 4 with a plurality of radiation detecting elements 7 arrayed thereon and a scan driver 15.

As illustrated in FIGS. 2 and 3, in the present embodiment, the plurality of radiation detecting elements 7 are arrayed so as to be two-dimensionally distributed on a surface 4a of the sensor substrate 4, and an area in which the plurality of radiation detecting elements 7 are arrayed (area surrounded by the dashed line in FIG. 2) is referred to as “radiation detection area P”. In the present embodiment, a plurality of scanning lines 5 and a plurality of signal lines 6 are arranged on the sensor substrate 4 so as to cross each other, and in each of small areas r defined by the scanning lines 5 and the signal lines 6, a radiation detecting element 7 is provided.

Also, as illustrated in FIGS. 2 to 4, bias lines 9 are connected to the respective radiation detecting elements 7. In the present embodiment, each bias line 9 is connected to a connection line 10, and for example, at the position indicated by A in FIG. 2, the connection line 10 and the respective signal lines 6 cross each other with a non-illustrated insulation layer therebetween. Also, a reverse bias voltage Vbias is applied to the respective radiation detecting element 7 from a bias power source 14 via the bias lines 9 and the connection line 10. In other words, a voltage applicator in the present invention is configured by the bias lines 9 and the bias power source 14.

Also, in each radiation detecting element 7, charge according to an amount of radiation received is generated inside the radiation detecting element 7.

Also, each radiation detecting element 7 is connected to a relevant signal line 6 via a TFT 8, which serves as a switching element. Also, as illustrated in FIG. 2, a pad 11 is provided at an end portion of each of the scanning lines 5, the signal lines 6 and the connection lines 10, etc., respective wirings from a non-illustrated flexible circuit substrate, etc., are connected to the respective pads 11, and the scanning lines 5, the signal lines 6, the connection lines 10, etc., are connected to non-illustrated electronic components (bias power source 14, etc.) provided on the back side of the sensor substrate 4.

In the scan driver 15, either an on-state voltage or an off-state voltage, which is supplied from a power source circuit 15a via a wiring 15c, is selected by a gate driver 15b and applied to respective lines L1 to Lx of the scanning lines 5. Then, each TFT 8 enters an off state when the off-state voltage is applied to the TFT 8 via the relevant scanning line 5, and shuts off conduction between the relevant radiation detecting element 7 and the relevant signal line 6 and causes charge to be accumulated inside the radiation detecting element 7. Also, each TFT 8 enters an on-state when the on-state voltage is applied to the TFT 8 via the scanning line 5, and causes the charge accumulated inside the radiation detecting element 7 to be released to the signal line 6.

The above-described configuration enables each small area r to accumulate and release an amount of charge, the amount corresponding to an intensity of radiation received. In the below, each of these small areas is referred to as “pixel r”.

Also, the signal lines 6 are connected to respective readout circuits 17 incorporated in a readout integrated circuit (ROIC) 16. Each readout circuit 17 is intended to read out image data based on an amount of charge released by each of relevant pixels, and includes an integration circuit 18, a correlated double sampling circuit 19, an analog multiplexer 21 and an A/D converter 20. Note that in FIGS. 3 and 4, a correlated double sampling circuit 19 is indicated as “CDS”. Also, in FIG. 4, illustration of an analog multiplexer 21 is omitted.

Note that although FIG. 3 indicates a sensor substrate 4 including a single readout integrated circuit 16 as an example, as illustrated in FIG. 9, a sensor substrate 4 may include a plurality of readout integrated circuits 16 and the plurality of readout integrated circuits 16 may be driven upon supply of power from different power source circuits.

In the present embodiment, as illustrated in FIG. 4, each integration circuit 18 includes an operational amplifier 18a, a capacitor 18b and a charge reset switch 18c, which are connected in parallel. Also, each of the signal lines 6 is connected to an inverting input terminal of the operational amplifier 18a of the relevant integration circuit 18, and a reference voltage V0 is applied to a non-inverting input terminal of the integration circuit 18. Therefore, the reference voltage V0 is applied to the respective signal lines 6.

Also, the charge reset switches 18c of the integration circuits 18 are connected to a later-described controller 22 and is controlled to be turned on or off by the controller 22. When a TFT 8 is turned on in a state in which the relevant charge reset switch 18c is off, charge released from the relevant radiation detecting element 7 flows into and accumulated in the relevant capacitor 18b, and a voltage value according to the amount of accumulated charge is output from an output terminal of the relevant operational amplifier 18a.

Here, when the charge reset switch 18c is turned on, the input side and the output side of the integration circuit 18 is short-circuited, whereby the charge accumulated in the capacitor 18b is released and the capacitor 18b is thus reset. Also, the integration circuits 18 are driven upon supply of power from a power supply section 18d.

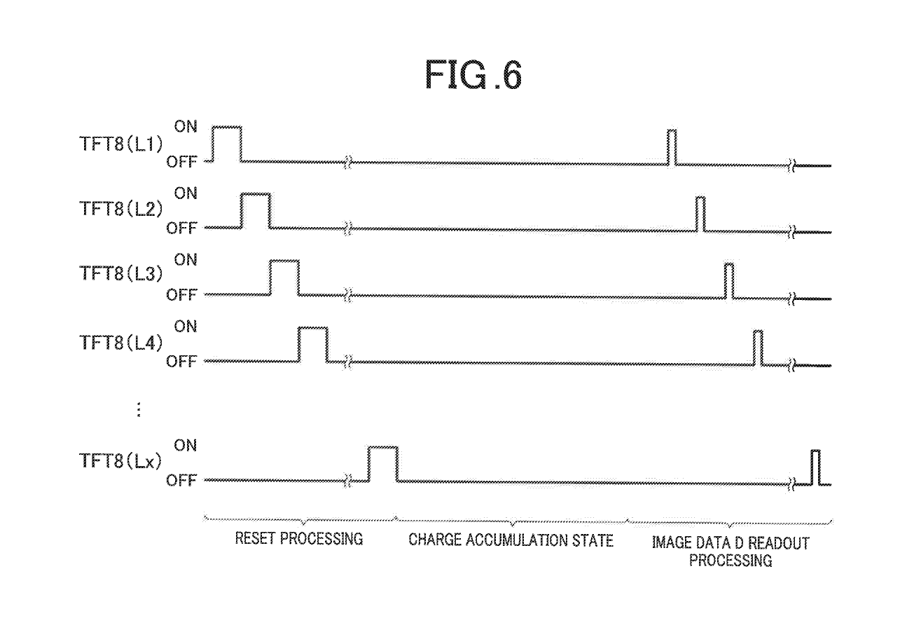

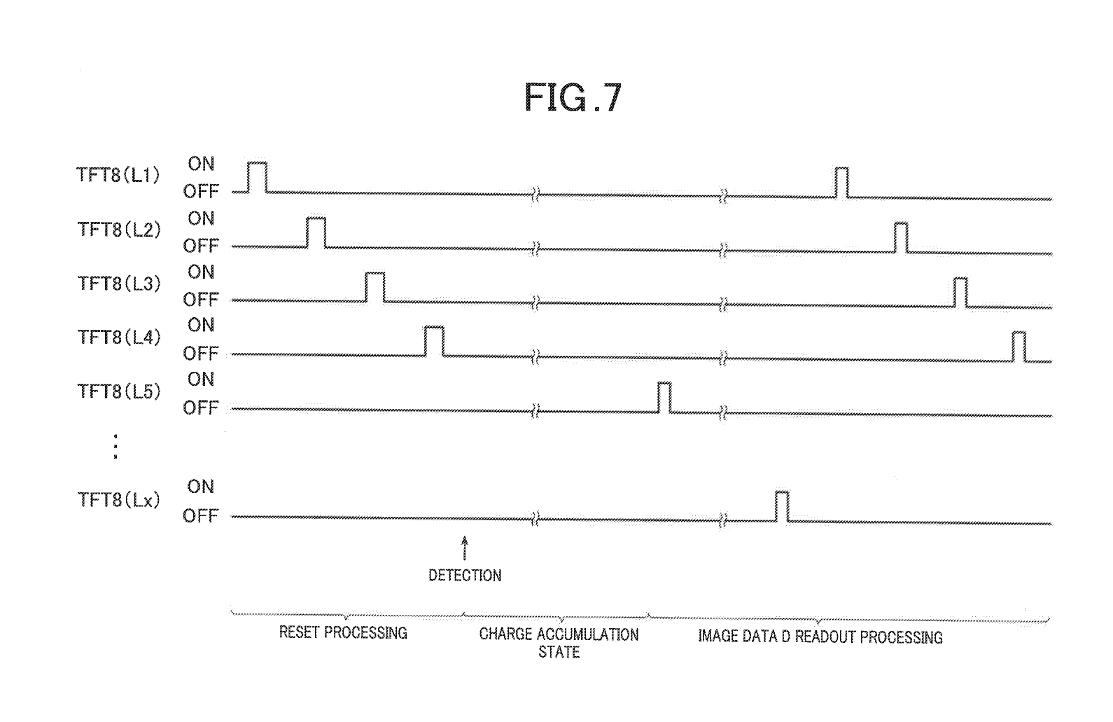

In processing for reading out image data D from each respective radiation detecting element 7 (see FIGS. 6 and 7 referred to later), which is performed after radiographing, as illustrated in FIG. 5, upon a first pulse signal Sp1 being transmitted from the controller 22 at a point of time of the charge reset switch 18c of the relevant integration circuit 18 being turned off, the relevant correlated double sampling circuit 19 holds a voltage value Vin output from the integration circuit 18 at that point of time.

Then, the on-state voltage is applied to a line Ln of the scanning lines 5 from the gate driver 15b. Then, upon the relevant TFTs 8 being turned on, charges are released to the signal lines 6 from the relevant radiation detecting elements 7 connected to the line Ln of the scanning lines 5 via the TFTs 8, the charges flow into the respective capacitors 18b of the readout circuits 17 via the signal lines 6, and the voltage values output from the relevant integration circuits 18 are thereby increased.

Upon a second pulse signal Sp2 being transmitted from the controller 22, the correlated double sampling circuit 19 holds a voltage value Vfi output from the integration circuit 18 at that point of time and outputs and thus reads out a difference Vfi-Vin between the voltage values as analog value image data D. Then, the output image data D are sequentially transmitted to the A/D converter 20 via the analog multiplexer 21, sequentially converted to digital value image data D by the A/D converter 20 and sequentially stored in a storage 23.

Then, as illustrated in FIG. 5, the on-state voltage is sequentially applied to respective lines L1 to Lx of the scanning lines 5 from the gate driver 15b (in FIG. 5, the on-state voltage is sequentially applied to the line Ln and a next line Ln+1 of the scanning lines 5) and the above-described processing is repeated, and as a result, image data D are read out from the respective radiation detecting element 7.

The controller 22 includes a computer including, e.g., a non-illustrated central processing unit (CPU), a read-only memory (ROM), a random access memory (RAM) and an input/output interface that are connected to a bus, and a field-programmable gate array (FPGA). The controller 22 may be configured by a dedicated control circuit.

Also, the storage 23 including, e.g., a static RAM (SRAM), a synchronous DRAM (SDRAM) and/or a NAND-type flash memory and an internal power source 24 are connected to the controller 22, and a communication section 30 for wireless or wired external communication via the aforementioned antenna 29 or the connector 27 is connected to the controller 22.

The controller 22 performs control such as control of operation of the scan driver 15 to perform processing for resetting the radiation detecting elements 7 or apply the off-state voltage to the TFTs 8 via the lines L1 to Lx of the scanning lines 5 from the gate driver 15b of the scan driver 15 for transition to a charge accumulating state, and control of operation of the scan driver 15, the readout circuits 17, etc., to perform processing for reading out image data D from the respective radiation detecting elements 7.

Also, the controller 22 can sequentially acquire a plurality of image data by repeating accumulation and release of charge by the respective pixels r and readout of the image data from the readout circuits 17 a plurality of times in a short period of time based on a single radiographing start instruction (for example, an operation of an exposure switch). The plurality of image data obtained as described above form frames forming a moving image.

Also, in the present embodiment, as described above, the controller 22 cause read-out image data D to be stored in the storage 23. Also, the controller 22 causes the communication section 30 to transmit the image data D to a non-illustrated image processing apparatus wirelessly or by wire via the antenna 29 or the connector 27 at a predetermined timing.

Radiographic image radiographic apparatuses are roughly divided into those for what is called a cooperation scheme, which each perform radiographing while transmitting and receiving signals and the like to or from (in cooperation with) a non-illustrated radiation apparatus that delivers radiation to the radiographic image radiographic apparatus, and those for a non-cooperation scheme, which each perform radiographing without transmitting and receiving signals and the like to or from (in no cooperation with a radiation apparatus.

The radiographic apparatus 1 according to the present embodiment can be configured as being of either of these types by changing control performed by the controller 22.

The controller 22 where the radiographic apparatus 1 is configured as one for the cooperation scheme, as illustrated in FIG. 6, sequentially applies the on-state voltage to the respective lines L1 to L of the scanning lines 5 from the gate driver 15b (see FIG. 3) of the scan driver 15 to perform processing for resetting the radiation detecting elements 7, before reception of radiation.

Then, upon a signal representing an effect that radiation is to be delivered being transmitted from a radiation apparatus, the controller 22 causes the off-state voltage to be applied to the respective lines L1 to Lx of the scanning lines 5 for transition to a charge accumulating state in which charges generated in the respective radiation detecting elements 7 as a result of reception of radiation are accumulated in the respective radiation detecting elements 7.

Then, upon an end of reception of the radiation, the on-state voltage is sequentially applied to the respective lines L1 to Lx of the scanning lines 5 from the gate driver 15b to perform processing for reading out image data D.

On the other hand, where the radiographic apparatus 1 is configured as one for the non-cooperation scheme, unlike the case of the cooperation scheme described above, the radiographic apparatus 1 cannot receive a signal representing an effect that radiation is to be delivered, from the radiation apparatus, and thus, detects a start of radiation by itself.

For processing for detecting a start of radiation, for example, any of the methods described in, e.g., Japanese Patent Laid-Open No. 2009-219538 and International Publication Nos. WO 2011/135917 and WO 2011/152093 can be employed, and for details, the above publications, etc., should be referred to.

Then, as illustrated in FIG. 7, the controller 22 in the case of the non-cooperation scheme sequentially applies the on-state voltage to the respective lines L1 to Lx of the scanning lines 5 from the gate driver 15b to perform processing for resetting the radiation detecting elements 7, before reception of radiation.

Then, upon a start of radiation, the controller 22 causes the off-state voltage to be applied to the respective lines L1 to Lx of the scanning lines 5 for transition to a charge accumulating state, and after an end of the radiation, the on-state voltage is sequentially applied to the respective lines L1 to Lx of the scanning lines 5 from the gate driver 15b to perform processing for reading out image data D.

As described above, the present invention is applicable to both radiographing in the cooperation scheme and radiographing in the non-cooperation scheme.

Also, the radiographic apparatus 1 is configured so as to after or before radiographing being performed in such a manner as described above (however, in a state in which no radiation is delivered to the radiographic apparatus 1), repeat a processing sequence up to processing for reading out image data D, which is illustrated in FIG. 6, etc., to perform processing for reading out image data of a dark charge image (hereinafter “dark charge image data O”).

Also, the controller 22 repeats accumulation and release of charges by the respective pixels and readout of dark charge image data O by the readout integrated circuit 16 a plurality of times before a start of radiographing, to sequentially acquire a plurality of dark charge image data O.

Note that the acquisition of dark charge image data may consistently be performed during the power of the radiographic apparatus 1 being on.

Inside each radiation detecting element 7, a dark charge (also referred to as, e.g., “dark current”) is consistently generated by thermal excitation caused by heat (temperature) of the heat radiation detecting element 7 itself, and an offset component generated by the dark charge is superimposed on image data D. Dark charge image data O is data corresponding to the offset component of the dark charge, and genuine image data D* attributable to charge generated inside the radiation detecting element 7 as a result of reception of radiation can be calculated by subtracting the dark charge image data O from the image data D according to expression (1) below and thereby performing offset correction of the image data D.

D*=D−O (1)

### [Noise Detector]

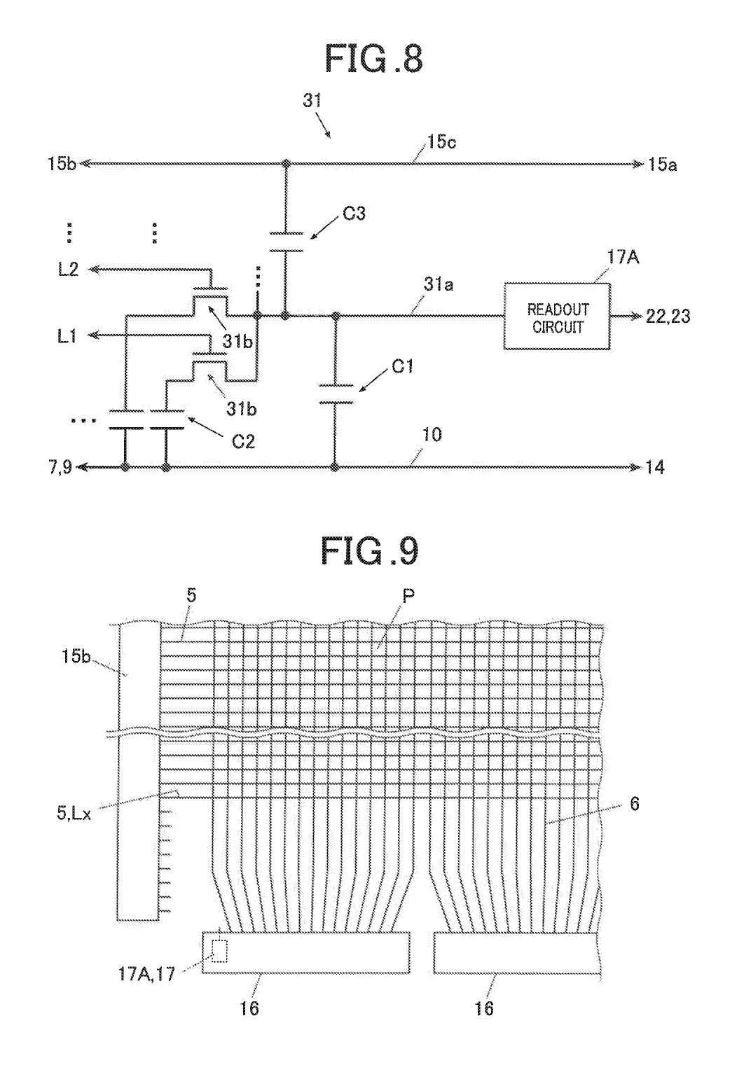

Next, a configuration, etc., of a noise detector in the above radiographic apparatus 1 will be described. FIG. 8 is a diagram illustrating an example configuration of a noise detector.

The radiographic apparatus 1 according to the present embodiment includes a noise detector 31. The noise detector 31 is intended to detect data corresponding to a noise component superimposed on image data D read out as a result of processing for reading out the image data D in such a manner as described above.

Note that the noise detector 31 may be provided, for example, on the front surface 4a side or the back surface side of the sensor substrate 4 (see FIG. 2) or can be provided on the aforementioned flexible circuit substrate. Note that the reference numeral at each of arrows in FIG. 8 referred to below represents a connection destination to which the relevant wiring is connected.

Note that a plurality of noise detectors 31 may be provided for one radiation detection area P.

As illustrated in FIG. 8, the noise detector 31 according to the present embodiment includes a correction signal line 31a, respective capacitors C1 to C3 and a readout circuit 17A connected to the correction signal line 31a. In the present embodiment, as the readout circuit 17A of the noise detector 31, a readout circuit 17 formed in the above-described readout integrated circuits 16 (see FIGS. 3 and 4) is used.

Therefore, as with the above-described readout circuits 17 (see FIGS. 3 and 4) for reading out image data D, the readout circuit 17A in the present embodiment includes, e.g., an integration circuit 18 and a correlated double sampling circuit 19 (illustration thereof is omitted in FIG. 8). Then, as in the case of the above-described signal lines 6, the reference voltage V0 is applied from the integration circuit 18 inside the readout circuit 17A to the correction signal line 31a.

Note that in the present embodiment, as the readout circuit 17A of the noise detector 31, for example, as illustrated in FIG. 9, a readout circuit 17 to which no signal line 6 is connected (for example, a readout circuit 17 at an end portion of a readout integrated circuit 16 is used, and although the illustration is omitted, the correction signal line 31a of the noise detector 31 is connected to the readout circuit 17 with no signal line 6 connected thereto.

Note that although the illustration is omitted in FIG. 9, the correction signal line 31a is arranged, for example, in parallel with a signal line 6 closest to the gate driver 15b, between the gate driver 15b and the signal line 6.

Also, in the present embodiment, the readout circuit 17A of the noise detector 31 detects data d₃₁ by performing processing that is similar to the above-described image data D readout processing by another readout circuit 17, and the detected data d₃₁ is digitalized by the A/D converter 20 and stored in the storage 23.

Note that for the readout circuit 17A of the noise detector 31, it is not necessarily necessary to use a readout circuit 17 provided inside the readout integrated circuit 16, and a readout circuit that is separate from the readout integrated circuit 16 can be provided.

Also, in the present embodiment, the readout circuit 17A of the noise detector 31 has a function that detects disconnection of the correction signal line 31a. More specifically, the readout circuit 17A monitors a width of fluctuation of the bias power source, which has been stated in the beginning, and if the fluctuation width falls below a predetermined value, determines that a disconnection occurs.

If a plurality of noise detectors 31 are provided for one radiation detection area P as mentioned above, it is possible that: each noise detector 31 can detect an abnormality, such as a disconnection, occurring in the relevant correction signal line 31a; in normal times, the controller 22 cause any of the plurality of noise detectors 31 to operate and causes the other noise detectors 31 to be in a non-operating state; and if the operating noise detector 31 detects an abnormality, the controller 22 stops the operation of the noise detector 31 and causes another noise detector 31 to operate.

With such configuration as above, even if an abnormality occurs in a noise detector 31, correction of image data D can be performed correctly.

From among the respective capacitors C1 to C3, as illustrated in FIG. 8, a first capacitor C1 is a capacitor that converts a difference in potential between the correction signal line 31a and the connection line 10 (or the relevant bias line 9) into charge. Also, a third capacitor C3 is a capacitor that converts a difference in potential between the correction signal line 31a and the wiring 15c that supplies the off-state voltage to be applied to the scanning lines 5 from the power source circuit 15a to the gate driver 15b in the scan driver 15 into charge.

Also, a second capacitor C2 is a capacitor that converts a difference in potential between the correction signal line 31a and the connection line 10 into charge. The second capacitor C2 is provided for each of the lines L1 to Lx of the scanning lines 5, and a switcher 31b is connected to each second capacitor C2, the switcher 31b switching between connection and non-connection between the second capacitor C2 and the correction signal line 31a.

Then, each switcher 31b is turned on/off by the on-state voltage or the off-state voltage applied to the switched lines L1 to Lx of the scanning lines 5. Therefore, upon the on-state voltage being applied to a certain scanning line 5, the respective TFTs 8 and the switcher 31b connected to the scanning line 5 are turned on, and upon the off-state voltage being applied to the scanning line 5, the respective TFTs 8 and the switcher 31b connected to the scanning line 5 are turned off.

In the present embodiment, as described above, each switcher 31b is turned on/off according to the relevant TFTs 8, which are switch elements connected to the same scanning line 5 to which the switcher 31b is connected. Then, in the present embodiment, as illustrated in FIG. 8, a number of sets of a capacitor C2 and a switch element 31b, the number being equal to the number of the scanning lines 5, are provided. Each switch element 31b can be configured by, for example, a TFT.

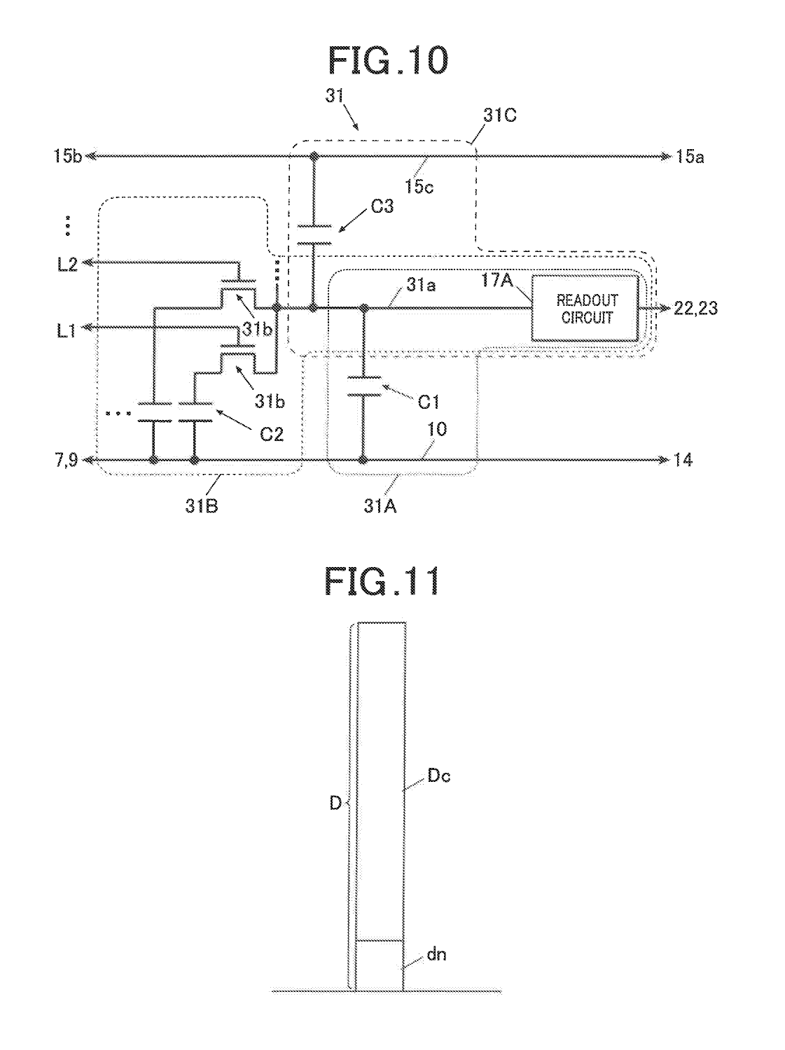

Here, in reality, as illustrated in FIG. 10, the configuration of the noise detector 31 illustrated in FIG. 8 is an assembly of noise detectors 31A, 31B, 31C, and the noise detectors 31A, 31B, 31C can be provided individually, and any two of noise detectors 31A, 31B, 31C can be combined.

The noise detector 31 is described in detail in Japanese Patent No. 5460103 mentioned above and thus Japanese Patent No. 5460103 should be referred to for details. Each of the noise detectors 31A, 31B, 31C will briefly be described below.

### [Noise Detector 31A]

In the noise detector 31A, charge of c1×(V0−Vbias) (c1 is an electrostatic capacitance of the first capacitor C1) is accumulated in the first capacitor C1; however, as illustrated in FIG. 21, voltage noise is generated in the reverse bias voltage Vbias and thus charge noise corresponding to the voltage noise is generated in the charge accumulated in the first capacitor C1. Also, charge noise that fluctuates in a phase that is exactly the same as that of the charge noise in the first capacitor C1 is generated in charge accumulated in each radiation detecting element 7. Here, the electrostatic capacitance c1 of the first capacitor C1 is set to be equal to an electrostatic capacitance of one radiation detecting element 7.

Then, in image data D reading processing, as illustrated in FIG. 5, each of a first pulse signal Sp1 and a second pulse signal Sp2 is transmitted to the respective correlated double sampling circuits 19 in the readout circuits 17 that read out image data D and the readout circuit 17A of the noise detector 31 simultaneously from the controller 22.

Therefore, data d₃₁ detected by the readout circuit 17A of the noise detector 31 includes noise data dₙ representing charge noise corresponding to the voltage noise in the reverse bias voltage Vbias, the noise data dₙ being superimposed on image data D read out by each readout circuit 17 at the same timing. In the below, the noise data dₙ is referred to as noise data d_{nA} attributable to voltage noise of a reverse bias voltage Vbias(t).

Then, each time the on-state voltage is applied to one of the lines L1 to Lx of the scanning lines 5 from the gate driver 15b and image data D is thereby read out in such a manner as described above in image data D readout processing (that is, each time first and second pulse signals Sp1, Sp2 are transmitted from the controller 22 to the correlated double sampling circuits 19 in readout processing for the respective lines L1 to Lx of the scanning lines 5), the noise detector 31A detects data d₃₁ containing noise data d_{nA} and stores the detected data d₃₁ in the storage 23.

### [Noise Detector 31C]

Next, before description of the noise detector 31B, the noise detector 31C will be described. As described above, the noise detector 31C includes the third capacitor C3, the wiring 15c that supplies an off-state voltage Voff from the power source circuit 15a to the gate driver 15b in the scan driver 15 (or the wiring 15c may be replaced with a scanning line 5 with the off-state voltage applied thereto. The same applies to the below.), the correction signal line 31a, and the readout circuit 17A.

Then, as with the aforementioned reverse bias voltage Vbias, voltage noise is generated temporally randomly in the off-state voltage Voff, and thus, charge noise corresponding to the voltage noise is generated in charge of c3×(V0−Voff) (c3 is an electrostatic capacitance of the third capacitor C3) accumulated in the third capacitor C3.

On the other hand, as described above, in image data D readout processing, charges accumulated inside the radiation detecting elements 7 connected to the scanning line 5 with the on-state voltage applied thereto from the gate driver 15b are released to the respective signal lines 6 via the relevant TFTs 8 brought in an on-state, and the released charges flow into the readout circuits 17.

In this case, the off-state voltage Voff is applied to several thousands of scanning lines 5 other than the scanning line 5 with the on-state voltage applied thereto. Then, as illustrated in FIG. 2 (see B in the figure), in each single signal line 6, a parasitic capacitance c is generated at each of parts crossing the respective scanning lines 5, and thus, in each crossing part, charge calculated as the product of the parasitic capacitance c and a potential difference V0−Voff between the reference voltage V0 and the off-state voltage Voff of the signal line 6 is accumulated. Then, as described above, voltage noise is also generated in the off-state voltage Voff.

Therefore, if the capacitance c3 of the third capacitor C3 is set to be equal to a total sum Σc of parasitic capacitances c formed at the respective crossing parts in which the scanning lines 5 cross the single signal line 6, data d₃₁ detected by the readout circuit 17A of the noise detector 31C contains noise data d_{nC} representing charge noise corresponding to the voltage noise in the off-state voltage Voff (total sum of charge noise in the respective crossing parts), the noise data d_{nC} being superimposed on image data D read out at a timing that is the same as a timing of detection of the data d₃₁.

Each time an on-state voltage is applied to one of the lines L1 to Lx of the scanning lines 5 from the gate driver 15b and image data D is thus read out in such a manner as described above in image data D readout processing (that is, each time first and second pulse signals Sp1, Sp2 are transmitted from the controller 22 to the correlated double sampling circuits 19 in readout processing for the respective lines L1 to Lx of the scanning lines 5), the noise detector 31C detects data d₃₁ containing noise data d_{nC} and stores the detected data d₃₁ in the storage 23.

### [Noise Detector 31B]

The image data D further contains, in addition to the above-described noise data d_{nA}, d_{nC}, noise data d_{nB}, which is an amount of fluctuation of charge noise corresponding to a difference between voltage noise in the reverse bias voltage Vbias at a point of time of switching the voltage applied to the TFTs 8 from the on-state voltage to the off-state voltage in processing for resetting the radiation detecting elements 7 (see FIGS. 6 and 7) and voltage noise in the reverse bias voltage Vbias at a point of time of switching the voltage applied to the TFTs 8 from the on-state voltage to the off-state voltage in subsequent image data D readout processing.

It is the noise detector 31B that detects data d₃₁ containing the noise data d_{nB}. In the noise detector 31B, a capacitance c2 of each second capacitor C2 is a capacitance that is equal to a parasitic capacitance of each of the radiation detecting elements 7 (or an average value of the parasitic capacitances of the radiation detecting elements 7) connected to a certain line Ln of the scanning lines 5, the certain line Ln being connected to the switcher 31b connected to the second capacitor C2. Then, when the on-state voltage is sequentially applied to the respective lines L1 to Lx of the scanning lines 5 from the gate driver 15b in such a manner as illustrated in FIGS. 6 and 7, the on-state voltage is also sequentially applied to the respective switchers 31b in the noise detector 31B simultaneously with the application of the on-state voltage to the lines L1 to Lx.

With such configuration as above, as illustrated in FIGS. 6 and 7, upon the voltage applied to the TFTs 8 and the switchers 31b of the noise detector 31B being switched from the on-state voltage to the off-state voltage at the time of processing for resetting the radiation detecting elements 7, voltage noise generated in the reverse bias voltage Vbias at that point of time is accumulated as charge noise in the third capacitor C3.

Then, upon the on-state voltage being applied to the TFTs 8 and the switchers 31b of the noise detector 31B in image data D readout processing, the applied on-state voltage being switched to the off-state voltage and data d₃₁ being detected by the readout circuit 17A of the noise detector 31B, the detected data d₃₁ contains noise data d_{nB} superimposed on the above-described image data D, after all.

In the present embodiment, the noise detector 31B detects data d₃₁ containing the above-described noise data d_{nB} superimposed on the read-out image data D and stores the detected data d₃₁ in the storage 23, respectively.

Also, as can be seen from the configuration illustrated in FIGS. 8 and 10, in the present embodiment, data d₃₁ containing the above-described respective noise data d_{nA}, d_{nB}, d_{nC} at the same time is detected. In the below, the description will be provided assuming that a total value of such noise data d_{nA}, d_{nB}, d_{nC} (d_{nA}+d_{nB}+d_{nC}) is noise data dₙ for each of the lines L1 to Lx of the scanning lines 5. However, as stated above, each of the noise detectors 31A to 31C can be configured independently so that the noise detectors 31A to 31C detect data d₃₁ containing noise data d_{nA} to d_{nC}, separately.

Then, as illustrated in FIG. 11, an effect of horizontal noise can be eliminated from image data D by subtracting noise data dₙ contained in data d₃₁ detected by the noise detector 31 at the same timing from image data D read out from the respective radiation detecting elements 7 in such a manner as described above, according to expression (2) below to calculate corrected image data D_{c}:

D_{c}=D−dₙ (2).

### [Offset Component in Readout Circuit Itself]

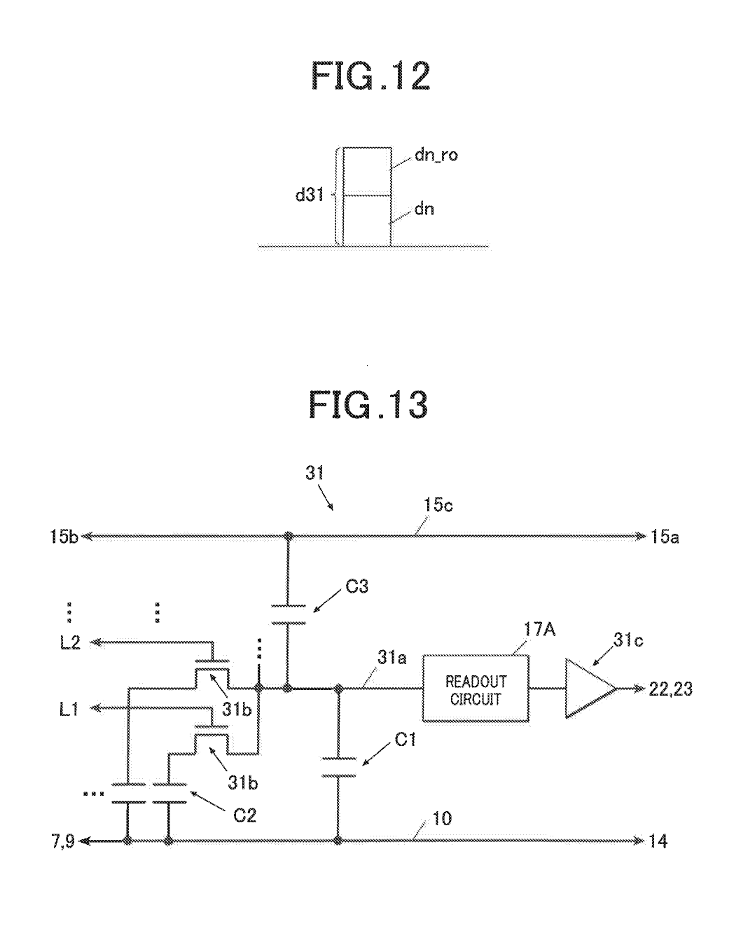

However, as stated in Japanese Patent No. 5460103 mentioned above (see the second embodiment in the same), although depending on the performance of the readout circuit 17A of the noise detector 31 (in the present embodiment, the performance is the same as that of the readout circuits 17 that read out image data D), as illustrated in FIG. 12, data d₃₁ detected by the noise detector 31 may contain an offset component dn_ro in the readout circuit 17A of the noise detector 31 itself, in addition to the above-described noise data dₙ (=d_{nA}+d_{nB}+d_{nC}).

In this case, a total value of the above-described noise data dₙ and the offset component dn_ro in the readout circuit 17A is detected as data d₃₁ by the readout circuit 17A of the noise detector 31, and where the image data D is corrected using such data d₃₁ in such a manner that is similar to the above, the corrected image data D_{c} can be expressed by expression (3) below:

D_{c}=D−d₃₁

∴D_{c}=D−(dₙ+dn_ro) (3)

Then, in Japanese Patent No. 5460103, for electrostatic capacitances c1 to c3 of respective capacitors C1 to C3 in a noise detector 31, those obtained by multiplying those illustrated in FIGS. 8 and 10 by W (W>1) are used, and as illustrated in FIG. 13, a multiplier 31c is provided on the output side of a readout circuit 17A of a noise detector 31 to multiply an output value from the readout circuit 17A by 1/W.

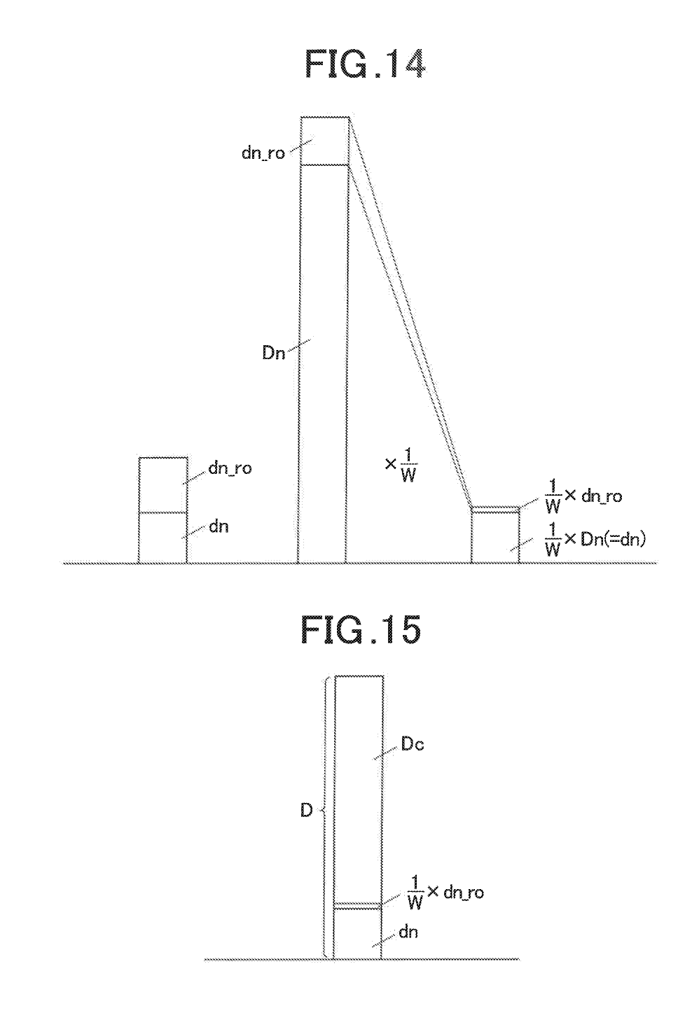

With such configuration as above, as illustrated in the center of FIG. 14, a total value of noise data Dₙ obtained by multiplying the above noise data dₙ by W and the offset component dn_ro (which is not multiplied by W) in the readout circuit 17A is output as data d₃₁ from the readout circuit 17A of the noise detector 31. Then, the data d₃₁ is multiplied by 1/W in the multiplier 31c, whereby the noise data Dₙ is multiplied by 1/W (1/W×Dₙ) and the offset component dn_ro in the readout circuit 17A are multiplied by 1/W (1/W×dn_ro). Then, the noise data Dₙ multiplied by 1/W is equal to the above noise data dₙ.

Therefore, as illustrated in FIG. 15, such configuration as above enables reducing an effect of the offset component dn_ro in the readout circuit 17A of the noise detector 31 on the image data D to 1/W while the noise data dₙ remains unchanged. Then, Japanese Patent No. 5460103 states that where a value of W is set to be sufficiently large, 1/W, which is the reciprocal of W, is very small and thus the effect of the offset component dn_ro in the readout circuit 17A of the noise detector 31 can be eliminated as much as possible from the corrected image data D_{c}.

### [Offset Component in Each Readout IC]

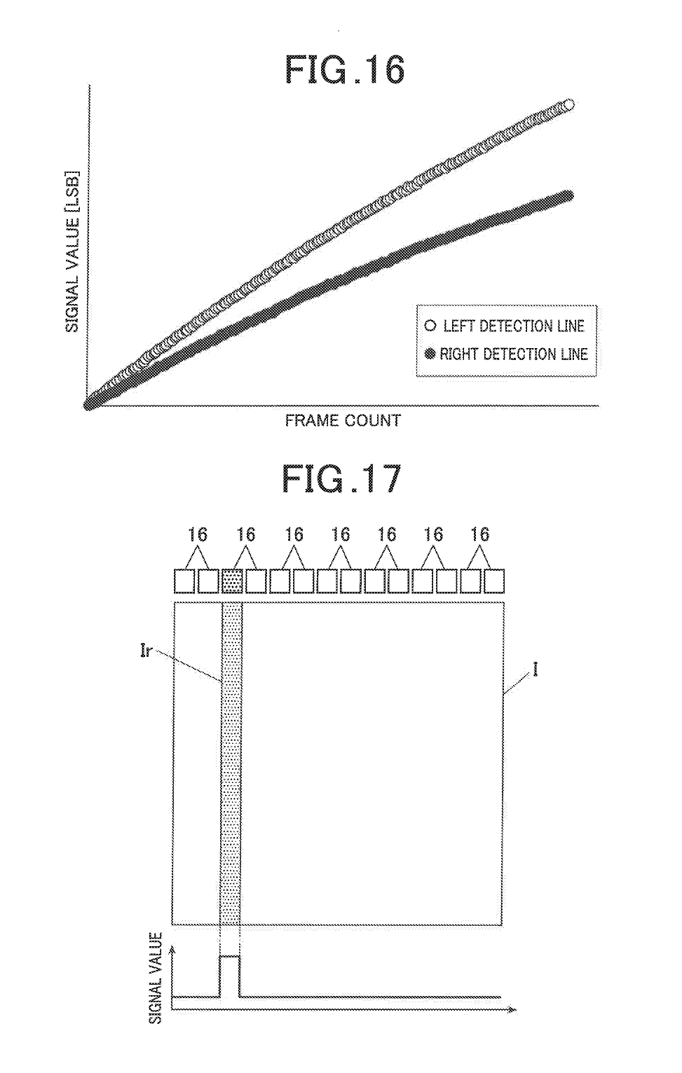

It is known that the above-described offset component dn_ro in the readout circuit 17A of the noise detector 31 (hereinafter simply referred to as offset component dn_ro in the noise detector 31), for example, as illustrated in FIG. 16, changes (increases) as radiographing of frames forming a moving image advances. Hereinafter, such temporal change of an offset component is referred to as “offset drift”. The offset drift occurs because a temperature of the readout circuit 17A of the noise detector 31 changes (increases) over time (each time radiographing of a frame is repeated).

Even if the effect of horizontal noise can be eliminated as a result of the radiographic apparatus 1 being configured as described above, because of this offset drift, an offset component to be subtracted vary depending on each frame included in the moving image.

Also, as with the readout circuit 17A of the noise detector 31, a temperature of each of the other readout circuits 17 for reading out image data D changes (increases) over time and an offset component dn_ro in such other readout circuit 17 changes (increases) temporally. However, since each of the other readout circuits 17 is different in circuit configuration from the readout circuit 17A of the noise detector 31, such changes are different from those of the noise detector 31.

Also, it is also known that: where a noise detector 31 is provided in each of a plurality of readout integrated circuits 16, a rate of temporal change (rate of increase: an inclination where expressed in a graph with a frame count or passage of time as the abscissa axis and a signal value as the ordinate axis) of an offset component dn_ro varies depending on each noise detector 31.

For example, where one radiation detection area P is divided into a plurality of band-like areas and different readout integrated circuits 16 are connected to the respective band-like areas, as illustrated in FIG. 16, a difference between signal values of offset components dn_ro on the right side and the left side becomes larger in the later frame, and thus, as replay of the moving image advances, for example, as illustrated in FIG. 17, an irregularity may occur in some band-like areas Ir in a displayed image I.

### [Characteristic Features of Radiographic Apparatus]

The controller 22 in the radiographic apparatus 1 according to the present embodiment has the following functions.

For example, the controller 22 has a function that builds a temporal change model of a signal value based on a plurality of dark charge image data O acquired before a start of radiographing.

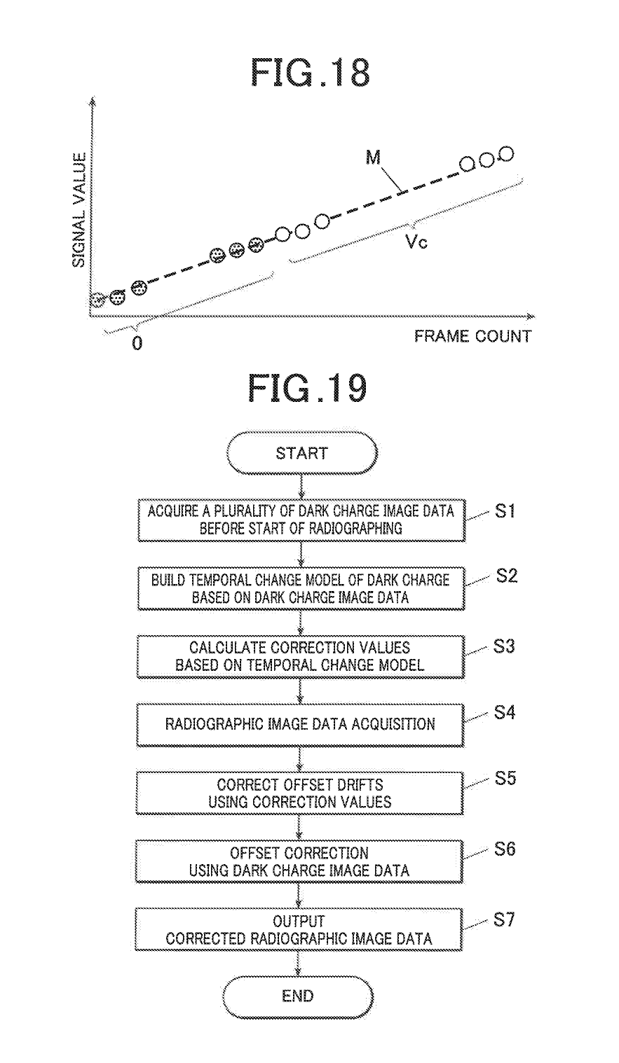

In the present embodiment, it is assumed that an offset component continues increasing linearly, and an inclination of a linear model (difference in signal value between two temporally consecutive frames) is estimated from a tendency of increase in respective signal values of the plurality of dark charge image data O acquired. As a result, as a temporal change model M, for example, as illustrated in FIG. 18, a linear graph with the frame count as the abscissa axis and the signal value as the ordinate axis is obtained.

Here, it is preferable that the controller 22 use those acquired for a period of 4 to 10 seconds before a start of radiographing from among the plurality of dark charge image data O acquired, for building the temporal change model. In this case, where a frame rate is set as 15 fps, the acquired dark charge image data O correspond to 60 to 150 frames.

Note that: an upper limit of time during which acquisition of dark charge image data O is repeated is set as 10 seconds from the perspective of wait time acceptable to a user; and if there is no problem in waiting for 10 seconds or more, the upper limit may be set as exceeding 10 seconds (over 150 frames acquired).

Also, the controller 22 has a function that calculates a plurality of correction values Vc based on the built temporal change model.

As illustrated in FIG. 18, the temporal change model M in the present embodiment is approximated by a linear line and thus a signal value f(x) for image data of an x-th frame can be expressed by expression (4) below.

f(x)=ax+b (4)

wherein

a is an inclination of a linear line model (difference in signal value between two temporally consecutive frames);

b is a signal value for dark charge image data O of a first frame.

According to expression (4), a signal value of dark charge image data O for any frame counted serially from the dark charge image data O of the first frame, can be calculated. This signal value can be used as a correction value Vc for each image data.

Here, it is preferable that a correction value Vc be calculated with one readout integrated circuit 16 as a unit, that is, for each group of pixels connected to the respective readout circuits 17 included in one readout integrated circuit 16.

Also, the controller 22 has a function that applies respective corresponding correction values Vc to image data of a plurality of radiographic images acquired during radiographing of a subject to correct the image data of the respective radiographic images.

More specifically, from a signal value of the pixels for each image data, a correction value Vc corresponding to the image data is subtracted. Consequently, an effect of an offset drift is eliminated in each radiographic image data and the respective radiographic image data thus contain an equal offset component.

Here, it is preferable that correction of image data be performed with one readout integrated circuit 16 as a unit, that is, for each group of pixels connected to the respective readout circuits 17 included in one readout integrated circuit 16.

Also, where the radiographic apparatus 1 has a function that performs binning, correction may be performed after binning is performed, but it is preferable that correction be performed before binning is performed.

Also, the controller 22 has a function that performs offset correction using a part of acquired dark charge image data O.

As a result of the controller 22 having such functions as above, the radiographic apparatus 1 according to the present embodiment can correct an offset component in each of frames forming a moving image with high accuracy even if an offset drift occurs in moving image radiographing.

Also, the controller 22 may be provided with a function that uses a predetermined number of dark charge image data O in reverse chronological order from latest dark charge image data O from among the plurality of dark charge image data O acquired, for building the temporal change model.

In such case, dark charge image data O acquired when the readout circuits 17 are in a state close to that at the time of radiographing are used for building the temporal change model, enabling more accurate correction.

Also, the controller 22 may be provided with a function that uses those acquired after driving (warming up) the readout integrated circuit 16 for a predetermined period of time (for example, around 30 seconds) from among the plurality of dark charge image data O acquired, for building the temporal change model.

It is known that: during a predetermined period of time from a start of driving of the readout integrated circuit 16, the temperature of the readout integrated circuit 16 sharply increases; and after passage of the predetermined period of time, the temperature increase becomes slow. Therefore, the provision of such function as above allows the degree of offset drift at the time of acquisition of dark charge image data O and the degree of offset drift at the time of acquisition of radiographic image data to be substantially equal to each other, enabling building a more accurate temporal change model and thus enabling more accurate correction.

Also, the controller 22 may be provided with a function that increases/decreases the number of dark charge image data O used for building the temporal change model, according to a period of time required for future radiographing of the subject.

With such configuration as above, increasing (decreasing) the number of dark charge image data O where radiographing time is long (short) enables building a temporal change model suitable for radiographing time, and thus enabling more accurate correction.

### [Moving Image Radiographing]

Next, a flow of moving image radiographing using the radiographic apparatus 1 according to the present embodiment will be described. FIG. 19 is a flowchart illustrating a flow of moving image radiographing using the radiographic apparatus 1 according to the present embodiment.

First, as illustrated in FIG. 19, a plurality of dark charge image data O is sequentially acquired before radiographing (step S1). For example, upon a user making a reservation of radiographing, such acquisition automatically starts. Then, the radiographic apparatus 1 builds a temporal change model of a signal value based on the plurality of dark charge image data O acquired (step S2) and calculates correction values based on the temporal change model (step S3).

In or after step S2, the user performs moving image radiographing (serial radiographing) to acquire image data of radiographic images of a subject (step S4). Then, the radiographic apparatus 1 applies correction values Vc to image data of radiographic images to correct an offset drift in the respective radiographic image data (step S5) and further performs offset correction of the radiographic image data using the dark charge image data O (step S6). Then, the radiographic apparatus 1 outputs the corrected image data to an external apparatus (step S7).

In a stationary radiographic apparatus, during moving image radiographing, a temperature of a readout integrated circuit 16 does not sharply increase and thus generally the problem of an offset drift does not occur so much. As reasons of the above, there are two main reasons (1) and (2) below:

(1) In the case of a stationary radiographic apparatus, a position at which such apparatus is installed is not changed (moved) because of the product characteristics thereof, and thus the apparatus is normally supplied with power via a wire cable. In this case, there is no need to worry about a remaining amount of power and thus readout circuits can consistently be in a driving state. As a result, temperatures of readout circuits 17 are maintained high, and fluctuation in temperature of the relevant readout integrated circuit 16 as a result of moving image radiographing being performed less likely occurs.
(2) A stationary radiographic apparatus is less limited in terms of size and weight, and thus, it is possible to bring a substance with a large thermal capacitance (for example, a metal block with a large volume or the like) into physical contact with readout circuits 17 or install a cooling mechanism (for example, a general air-cooling or water-cooling mechanism, a Peltier element, a cooling medium or the like) in the readout circuits 17 to suppress temperature fluctuation itself.

On the other hand, the radiographic apparatus 1 according to the present embodiment is supplied with power from an internal battery. In the case of power supply from a battery, it is difficult to consistently keep the power on from the perspective of securing usable time for a user, and thus, when radiographing is not performed immediately, the power is turned off, resulting in decrease in temperature of the readout integrated circuit 16.

Also, a substance with a large thermal capacitance and a cooling mechanism both increase the bulk and weight of the radiographic apparatus 1, and thus as in the above, it is difficult to provide such substance or such cooling mechanism to a portable radiographic apparatus, which requires a JIS-standard bulk (thickness) and light weight.

Therefore, the radiographic apparatus 1 according to the present embodiment cannot suppress an increase in temperature of the readout circuits at the time of moving image radiographing, resulting in occurrence of an offset drift such as described above. Even if a correction technique for a conventional stationary radiographic apparatus is used for such radiographic apparatus 1, no sufficient correction can be performed.

However, conditions for acquisition of dark charge image data O are set as described above, that is, a substantial increase in acquisition time and number of acquired image data relative to those of conventional techniques enables enhancement in accuracy of a temporal change model. As a result, accuracy of calculated correction values Vc is also enhanced, and even if an offset drift occurs when moving image radiographing is performed, an offset component in each of frames forming a moving image can be corrected with high accuracy.

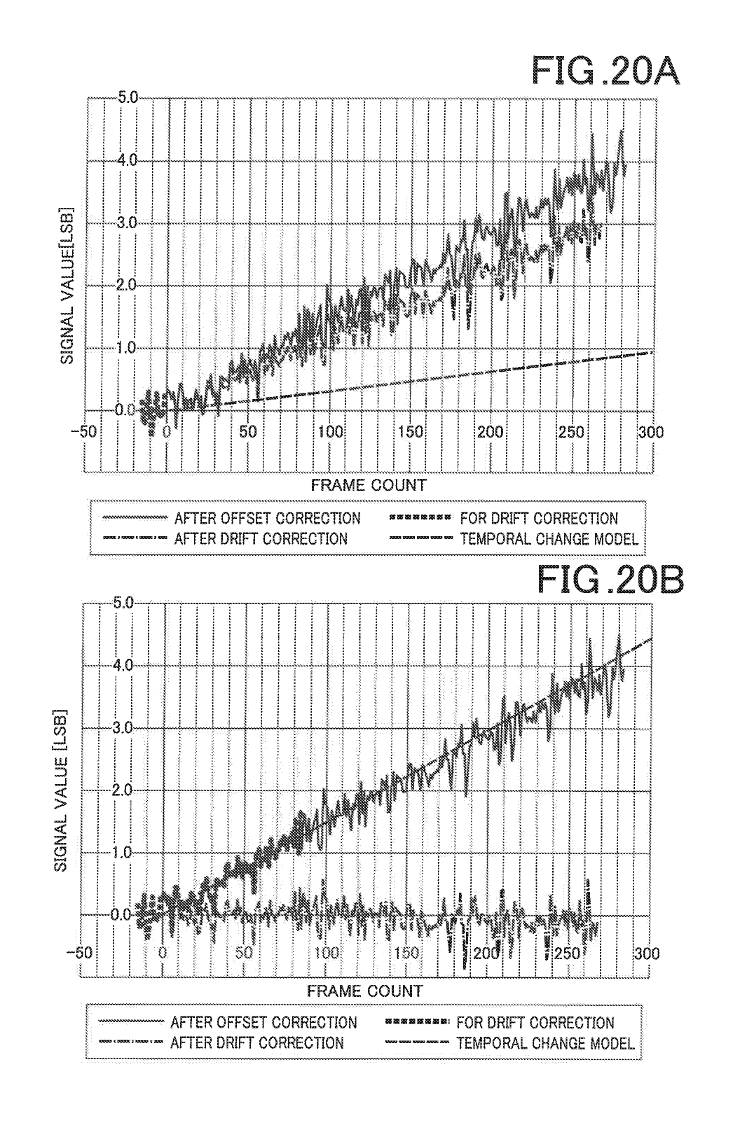

In other words, in the conventional correction method, as illustrated in FIG. 20A, the number of dark charge image data used for building a temporal change model is small (here, 16), and thus, the built temporal change model often diverges from an actual signal value increase. As a result, even if correction is performed using correction values calculated from the temporal change model, an effect of an offset drift cannot be eliminated so much.

On the other hand, in the correction method according to the present embodiment, as illustrated in FIG. 20B, the number of dark charge image data use for building a temporal change model is several times larger (here, 100) than that of the conventional technique, and thus, the built temporal change model substantially matches an actual signal value increase. As a result, an effect of an offset drift can sufficiently be eliminated by performing correction using correction values Vc calculated from the temporal change model.

Corrected moving image data is subjected to a dynamic analysis using an application, for example, chest dynamic imaging (CDI) or chest functional imaging (CFI).

CDI is an application for obtaining an index for evaluating movement of a radiographing subject region, and upon start-up of CDI, first, a moving image is acquired and shape-related information relating to a shape of a moving/deforming part in a two or more frame images included in the acquired moving image is recognized. Then, based on the recognized shape-related information, a deformation evaluation value relating to deformation of the moving/deforming part according to passage of time is calculated and output. At this time, as necessary, statistics on a plurality of deformation evaluation values calculated for two or more frame images in the moving image are also calculated and output.

CFI is an application for obtaining an index for evaluating the bloodstream flow or the ventilatory function (in the case of the lung) of a radiographing subject region, and upon start-up of CFI and selection of bloodstream analysis, first, a moving image is acquired, and bloodstream signal components are extracted from a series of frame images forming the acquired moving image, using a frequency filter and/or techniques such as average waveform and/or machine learning. Then, processing for calculating differences in extracted bloodstream signal component between respective frames is performed to calculate an amount of change in bloodstream signal component. Then, based on the amount of change in bloodstream signal component, a feature amount (e.g., a speed, a size and/or a direction) of the bloodstream is calculated.

On the other hand, upon start-up of CFI and selection of a ventilation function, first, a moving image is acquired, each of a series of frame images forming the acquired moving image is divided into regions according to a predetermined division method. Then, an average signal value is calculated for each of divisional regions resulting from the division. This processing is performed for each frame image, and average signal values for corresponding divisional regions in the respective frames are acquired as chronological data (in amplitude and cycle).

In such dynamic analysis, conventionally, an artifact due to an effect of an offset drift is generated; however, if a dynamic analysis is performed after elimination of the effect of an offset drift using the correction method according to the present embodiment, generation of an artifact can be prevented.

Next, specific examples of the above-described embodiment will be described.

Under the below conditions, acquisition of dark charge image data O and moving image radiographing were performed, a temporal change model was built to eliminate an effect of an offset drift from the moving image data, and a dynamic analysis was performed. The moving images after the analysis are present invention examples 1 to 4.
Panel configuration: AeroDR fine (manufactured by Konica Minolta, Inc.)
Radiographing sequence: moving image radiographing mode
Temporal change model: linear extrapolation
Time of acquisition of dark charge image data O: 4, 6.6, 8, 10 seconds (number of dark charge image data acquired: 60, 99, 120, 150)
Dark charge image data used for offset correction: recently acquired 16 dark charge image data
Warm-up of readout integrated circuit 16: 30 seconds
Application used for dynamic analysis: CFI (chest functional imaging: ventilation function)

Also, using the same panel, under conditions that are the same as above except the below change in number of dark charge image data O acquired, acquisition of dark charge image data O and moving image radiographing were performed and offset correction of the moving images was performed. Corrected moving images are comparative examples 1 to 4.
Time of acquisition of dark charge image data O: 1, 3, 11, 14 seconds (number of dark charge image data O acquired: 15, 45, 165, 210)

Whether or not there is a correction remainder that was not completely corrected was determined for each of moving images according to present invention examples 1 to 4 and comparative examples 1 to 4, and if the correction remainder is one that does not affect diagnosis, the relevant example was determined as good, and if the correction remainder is one that adversely affects diagnosis, the relevant example was determined as poor.

Also, time of acquisition of data for correction was measured, and if the time is a length of wait time acceptable to a user, the relevant example was determined as good, and if the time is a length of wait time exceeding an acceptable limit for a user, the example was determined as poor.

Also, as comprehensive evaluation, if evaluations of “correction remainder” and “user wait time” are both “good”, the example was determined as “good”, and if evaluations of “correction remainder” and “user wait time” are not both “good” (at least one of the evaluations is “poor”), the relevant example was determined as “poor”.

Results of the determination are indicated in Table I below.

TABLE I

| | Time of | | | |
|---|---|---|---|---|
| | acquisition of | | | |
| | dark charge | | | |
| | image for | | | |
| | correction | Correction | User wait | Comprehensive |
| | [sec] | remainder | time | evaluation |
| Comparative | 1 | Poor | Good | Poor |
| example 1 | | | | |
| Comparative | 3 | Poor | Good | Poor |
| example 2 | | | | |
| Present | 4 | Good | Good | Good |
| invention | | | | |
| example 1 | | | | |
| Present | 6.6 | Good | Good | Good |
| invention | | | | |
| example 2 | | | | |
| Present | 8 | Good | Good | Good |
| invention | | | | |
| example 3 | | | | |
| Present | 10 | Good | Good | Good |
| invention | | | | |
| example 4 | | | | |
| Comparative | 11 | Good | Poor | Poor |
| example 3 | | | | |
| Comparative | 14 | Good | Poor | Poor |
| example 4 | | | | |

Comprehensive evaluations of present invention examples 1 to 4 were all “good”, and comprehensive evaluations of comparative examples 1 to 4 were all “poor”.

In other words, where the time of acquisition of dark charge image data O is less than 4 seconds (number of acquired dark charge image data O: less than 60), an artifact was generated in the moving image because of failure to build a sufficiently accurate temporal change model. It has been found from the tests that acquisition of 60 or more dark charge image data O enables obtainment of a satisfactory correction range.

Also, where the time of acquisition of dark charge image data O exceeded 10 seconds (number of acquired dark charge image data O: over 150), it was determined that many of users cannot accept the length of wait time before moving image radiographing.

Although the present invention has been described in detail above based on an embodiment and examples, the present invention is not limited to the embodiment and examples described above and can be altered without departing the spirit of the invention.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2018-029254, filed on 22 Feb. 2018, is incorporated herein by reference in its entirety.

What is claimed is:

1. A portable radiographic apparatus comprising:
a radiation detector including a substrate and a plurality of pixels capable of accumulating and releasing an amount of charge, the amount corresponding to an intensity of radiation received, the plurality of pixels being provided so as to be two-dimensionally distributed on a surface of the substrate;
a readout integrated circuit that reads out image data based on the amount of charge released from each of the pixels; and
a hardware processor that
sequentially acquires a plurality of image data by repeating charge accumulation and release by the pixels and image data readout by the readout integrated circuit a plurality of times,
builds a temporal change model of a signal value based on image data of a plurality of dark charge images acquired before a start of radiographing,
calculates a plurality of correction values based on the built temporal change model, and
applies the respective corresponding correction values to image data of a plurality of radiographic images acquired during radiographing of a subject to correct the image data of the respective radiographic images.

2. The portable radiographic apparatus according to claim 1, wherein the hardware processor uses image data acquired for a period of 4 to 10 seconds before a start of radiographing from among the image data of the plurality of dark charge images acquired, for building the temporal change model.

3. The portable radiographic apparatus according to claim 2, wherein the hardware processor uses a predetermined number of image data in reverse chronological order from latest image data from among the image data of the plurality of dark charge images acquired, for building the temporal change model.

4. The portable radiographic apparatus according to claim 3, wherein the hardware processor uses image data acquired after driving the readout integrated circuit for a predetermined period of time from among the image data of the plurality of dark charge images acquired, for building the temporal change model.

5. A portable radiographic apparatus comprising:
a radiation detector including a substrate and a plurality of pixels capable of accumulating and releasing an amount of charge, the amount corresponding to an intensity of radiation received, the plurality of pixels being provided so as to be two-dimensionally distributed on a surface of the substrate;
a readout integrated circuit that reads out image data based on the amount of charge released from each of the pixels; and
a hardware processor that
sequentially acquires a plurality of image data by repeating charge accumulation and release by the pixels and image data readout by the readout integrated circuit a plurality of times,
builds a temporal change model of a signal value based on image data of a plurality of dark charge images acquired before a start of radiographing,
calculates a plurality of correction values based on the built temporal change model, and
applies the respective corresponding correction values to image data of a plurality of radiographic images acquired during radiographing of a subject to correct the image data of the respective radiographic images,
wherein the hardware processor can increase and decrease a number of the image data of the dark charge images used for building the temporal change model, according to a length of time required for future radiographing of the subject to be performed.

6. A radiographic image correction method comprising:
sequentially acquiring image data of a plurality of dark charge images by repeating image data generation by a radiographic apparatus capable of generating image data of a radiographic image according to radiation received, a plurality of times before a start of radiographing;
building a temporal change model of a signal value based on the acquired image data of the plurality of dark charge images;
calculating a plurality of correction values based on the built temporal change model;
sequentially acquiring image data of a plurality of radiographic images by repeating image data generation by the radiographic apparatus a plurality of times during radiographing of a subject; and
applying the respective corresponding correction values to the acquired image data of the plurality of radiographic images to correct the image data of the respective radiographic images.
